(19) United States
(12) Patent Application Publication
(10) Pub. No.: US 2009/0275873 A1
(43) Pub. Date: Nov. 5, 2009

(54) CIRCULAR KNIT FABRIC FOR USE IN COMPRESSION THERAPY

(75) Inventor: Karl ACHTELSTETTER, Dasing-Wessiszell (DE)

Correspondence Address: Fleit Gibbons Gutman Bongini & Bianco PL, 21355 EAST DIXIE HIGHWAY, SUITE 115, MIAMI, FL, 33180, US

(73) Assignee: Julius Zorn GmbH, Aichach (DE)

(21) Appl. No.: 12/428,128

(22) Filed: Apr. 22, 2009

(30) Foreign Application Priority Data
May 2, 2008 (DE) 10 2008 021 998.3

(51) Int. Cl. A61F 13/00; A61L 15/00

(52) U.S. Cl. 602/76

(57) ABSTRACT

The present invention relates to a circular knit fabric for use in compression therapy of lymphedemas, having a surface with a patterned structure with protuberances (1) which, when in contact with the body part to be treated with compression therapy, exert pressure on the surface of the body part and which, when the body part moves, produce a massaging effect. To create such a circular knit fabric that is comfortable to the wearer and that, when worn, exerts a sufficiently high compressive pressure on the body part to be treated and, at the same time, improves lymph drainage, the invention proposes that the structured surface have a regular pattern in the form of chambers (2) that are bounded by protuberances (1), with the chambers (2a, 2b, 2c) that are adjacent to each other being connected to each other by means of connecting channels (3) in the longitudinal direction (L) of the circular knit fabric.

### CROSS REFERENCE TO RELATED APPLICATION

This application claims priority under 35 U.S.C. §119 to German Patent Application No. 10 2008 021 998.3 filed May 2, 2008, the entire contents of which are incorporated herein by reference.

### FIELD OF THE INVENTION

The present invention relates to a circular knit fabric for use in compression therapy and to the use of this circular knit fabric in compression therapy for lymphedemas or ulcus cruris venosum or in scar therapy as well as to a compression article produced from this type of circular knit fabric.

Lymphedema is the chronic swelling of body extremities. The swelling is caused by an inadequate transport of lymphatic fluid and the resultant accumulation of free protein-containing fluid in the interstitial tissue. A special form of lymphedema is lipedema, a disorder of fat distribution which leads to the chronic accumulation of fat especially in the thigh and hip region.

### BACKGROUND OF THE INVENTION

To treat lymphedemas and lipedemas, decongestive therapies are carried out. This involves manual or surgically aided lymph drainage and/or compression therapy with compression bandages or compression stockings. Frequently, a combination of lymph drainage and compression therapy is used. In manual lymph drainage, the lymph transport is stimulated by means of a special massage technique, and tissue indurations are softened. To ensure that the edema does not recur after lymph drainage, a compression treatment by means of compression bandages or compression stockings is carried out.

In compression therapy, flat knit compression articles are generally used. In the prior art, for example, flat knit compression stockings are known which are knitted stitch by stitch to follow the contour of the body. To create a compression stocking, the flat knit fabric is sewn together by means of a flat elastic seam. In contrast to circular knit fabrics which allow the creation of seamless stockings, flat knit fabrics generally have a more uniform compressive effect over an area on which they are worn, which is required in treating edemas by means of compression therapy. A strong compression stocking that is produced from a flat knit fabric does not yield to the edema, and the movement of the body extremities that are being treated leads to a high working pressure which leads to an optimum compression of the tissue.

DE 296 18 426 U1 discloses a structured support surface for compression bandages, in particular for use in the treatment of edemas, which is comprised of a basic body on which substantially uniform protuberances are integrally arranged. As the patient moves, the elevations are meant to produce a massaging effect (micromassage) relative to the body surface of the body part to be treated, which aims at softening the fibrotic structures (indurations of the connective and fatty tissue and protein inclusions) in the subcutaneous tissue and at activating the microcirculation and, at the same time, at promoting lymph drainage.

The flat knit compression articles in the form of compression stockings and sleeves generally used in the compression treatment of edemas are not especially popular among the patients since they are less comfortable and they look less attractive than the circular knit compression articles. Especially the seam which is unavoidable when flat-knit fabrics are used is considered uncomfortable and gives it an unpleasant look.

Thus, the problem to be solved by the present invention is to make available a knit fabric for use in compression therapy of lymphedemas and lipedemas in the form of a more comfortable circular knit fabric which, when worn, exerts a sufficiently high compressive pressure on the body part to be treated and, at the same time, promotes lymph drainage.

### SUMMARY OF THE INVENTION

This problem is solved with a circular knit fabric with the features of claim 1. Preferred embodiments and uses of this circular knit fabric follow from the dependent claims.

### BRIEF DESCRIPTION OF THE FIGURES

The invention will be explained in greater detail below with reference to the enclosed drawings which illustrate a practical example. In the drawings,

FIG. 1 shows a knitting stitch pattern of a first embodiment of the circular knit fabric according to the present invention (FIG. 1a) and computer-generated pattern images of this first embodiment of the circular knit fabric for the knitting thread (FIG. 1b) and the weft thread (FIG. 1c); and

FIG. 2 shows the knitting stitch pattern of a second embodiment of the circular knit fabric according to the present invention (FIG. 2a) and a computer-generated pattern image of this second embodiment for the weft thread (FIG. 2b).

### DETAILED DESCRIPTION OF THE INVENTION

The circular knit fabric according to the present invention is a basic knit-purl knit which is made with a knitting thread that forms tuck loops to form a regular pattern. The tuck loops cause the knit to contract in the longitudinal direction L of the circular knit. In the basic circular knit, an elastic weft thread is floated with an offset of 1:1. The weft thread is preferably made of an elastic fiber, in particular elastodiene, elastane or elastane fibers covered with polyamide. The knitting thread is preferably an elastane fiber covered with polyamide, polyester, cotton, wool, silk or any other textile material (that can be combined with elastic fibers).

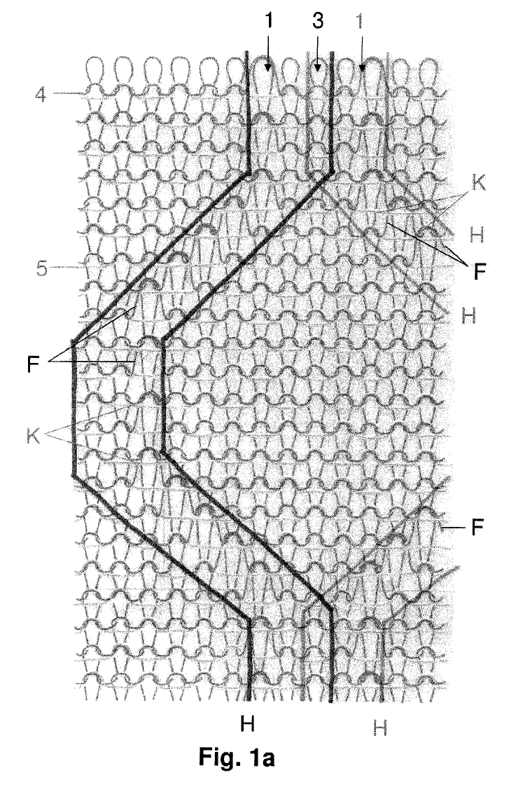

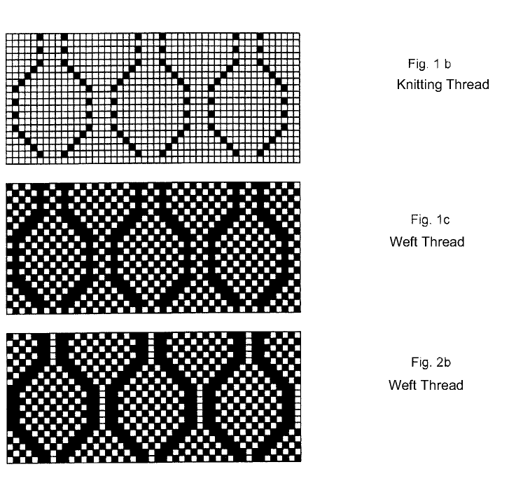

FIG. 1 schematically shows a first embodiment of a circular knit fabric according to the present invention in the form of a knitting stitch pattern (FIG. 1a) and in the form of a computer-generated pattern image (FIGS. 1b and FIG. 1c). In this first practical example of the circular knit fabric according to the present invention, the weft thread 5, at the points at which the knitting thread 4 forms tuck loops F, is not bound into the knit fabric but instead the weft thread floats. Due to the floating K of this weft thread 5, the weft thread causes the knit to contract across the area of the floating thread path along the width, i.e., in the circumferential direction of the circular knit fabric, thus leading to the compression desired.

The thus knitted circular fabric forms a surface having a patterned structure with protuberances 1, said protuberances being located in the areas of the knit fabric in which the weft thread is floating. In the areas in which the weft thread 5 is floating K, protuberances 1 are formed. In the computer-generated pattern images of FIGS. 1b and 1c, one can see the fill pattern with the protuberances 1. The protuberances 1 enclose chambers 2, within which the knit fabric has no protuberances. Chambers 2a, 2b that are adjacent to each other in the longitudinal direction L of the circular knit fabric are connected to each other by connecting channels 3, and these connecting channels 3 are also areas in which there are no protuberances in the knit fabric. In FIG. 1a, the protuberances are indicated by the aid lines H which only serve for clarification purposes and which are not part of the knit structure.

The chambers 2 are diamond-shaped or, as in the pattern images of FIGS. 1b and 1c, honeycomb-shaped. Because of the oblique tuck loops F in this pattern, it is possible to form tuck loops in every stitch row since they are again knit together with a stitch in the next stitch row. In the vertical direction of the pattern, a tuck loop can be formed only in every second stitch row since each tuck loop subsequently has to be knit to form a stitch. As a result, the floating weft thread is eliminated.

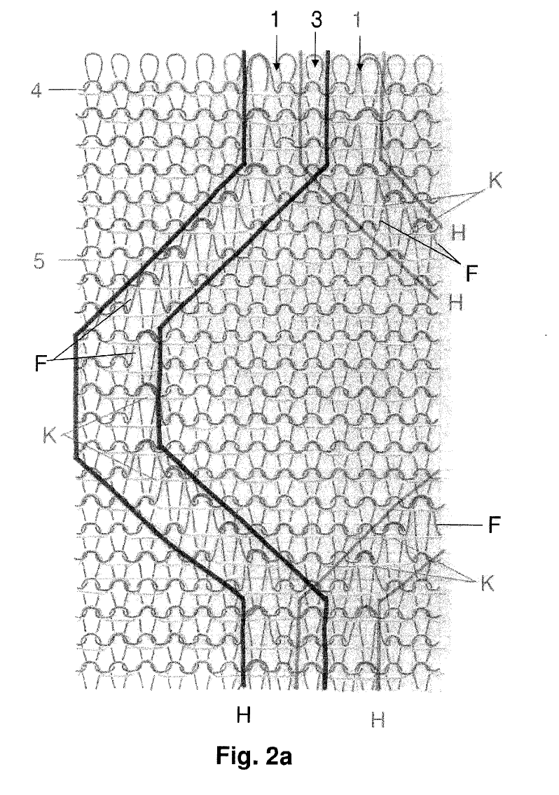

In the embodiment of the circular knit fabric shown in FIG. 2, the connecting channels are even more pronounced. Compared to the embodiment shown in FIG. 1, the plain knit differs in the vertical direction. Here, the float K of the weft thread 5 is laterally offset by one needle. The immediately following binding point is also laterally offset by one needle so that a float K over two needles is formed. In the narrow area between the thus formed protuberances 1 which form the connecting channels 3 between the chambers 2 that are bounded by the protuberances 1, the weft threads 5 in each row are bound in one above the other. The computer-generated pattern images for the knitting thread (FIG. 1b) are identical for both embodiments. The pattern images for the weft thread differ. In the practical example shown in FIG. 2, the diamond-shaped pattern which is formed by the protuberances 1 is more pronounced than in the practical example shown in FIG. 1 (see FIGS. 2b and 1c).

When a circular knit fabric is worn on a body part, the protuberances 1 exert pressure on the surface of the body part. As the body part moves, a massaging effect is added since the protuberances 1 move relative to and along the surface of the body part, thereby massaging it. This massaging effect promotes lymph drainage. The lymphatic fluid, the flow of which is stimulated by the massaging effect, can flow in the longitudinal direction of the circular knit fabric (metaphorically speaking) through the connecting channels 3 that connect the chambers 2 with one another and through the chambers 2 and, as a result, can be transported to the kidneys. Thus, because of the massaging effect and the flow of the lymphatic fluid through the chambers in the longitudinal direction L of the circular knit fabric, lymph drainage takes place. A compression article produced from a circular knit fabric according to the present invention is worn so that the right side of the fabric is in contact with the skin. Prior-art compression articles are normally worn the other way round. On the right side of the fabric, the pattern of the circular knit fabric is most pronounced and therefore is able to produce a stronger massaging effect on the skin.

The circular knit fabric according to the present invention can be used to produce compression articles, for example, compression stockings or compression sleeves, which can be used in compression therapy under high working pressure, for example, in compression therapy for lymphedemas or ulcus cruris venosum or in the treatment of scars. Circular knit fabrics according to the present invention can also be used in the treatment of skin alterations that are caused by ulcers and in the treatment of fibroses. Compression articles produced from a circular knit fabric according to the present invention can therefore be used in compression therapy under high working pressure in which the entire body, i.e., especially all extremities, can be treated with such compression articles.

1. A circular knit fabric for use in compression therapy, having a surface with a patterned structure with protuberances which, when in contact with the body part to be treated with compression therapy, exert pressure on the surface of the body part and which, when the body part moves, produce a massaging effect, wherein the structured surface has a regular pattern in the form of chambers which are bounded by the protuberances, with the chambers that are adjacent to each other being connected by connecting channels in the longitudinal direction of the circular knit fabric.

2. The circular knit fabric as in claim 1, wherein the chambers are diamond-shaped or honeycomb-shaped.

3. The circular knit fabric as in claim 1, wherein the fabric consists of a plain knit with a knitting thread which forms tuck loops and on which an elastic weft thread is floated with an offset of 1:1.

4. The circular knit fabric as in claim 3, wherein at the points at which the knitting thread forms tuck loops, the weft thread is not bound into the knit.

5. The circular knit fabric as in claim 4, wherein in the vertical direction of the knit, the knitting thread forms tuck loops only in every second row and that the float of the weft thread as well as the immediately following binding point is laterally offset by one needle so that the weft thread floats over two needles.

6. The use of a circular knit fabric as in claim 1 in compression therapy of lymphedemas or ulcus cruris venosum or in the treatment of scars.

7. A compression article, in particular a compression stocking or compression sleeve, for use in compression therapy and produced from a circular knit fabric as in claim 1.

8. A method of treating lymphedemas, ulcus cruris venosum or scars comprising the steps of forming a compression article from a circular knit fabric as in claim 1 and positioning the compression article around a portion of a body of a patient.
